(19) United States
(12) Patent Application Publication
(10) Pub. No.: US 2022/0084680 A1
(43) Pub. Date: Mar. 17, 2022

(54) PROVISION OF A STROKE CLASSIFICATION AND COORDINATION OF EMERGENCY SERVICES FOR POTENTIAL STROKE PATIENTS

(71) Applicant: Siemens Healthcare GmbH, Erlangen (DE)

(72) Inventors: Walter Schmid, Seefeld (DE); Sebastian Eckl, Forchheim (DE); Britta Kreuzer, Nuernberg (DE); Hanno Herrmann, Nuernberg (DE)

(73) Assignee: Siemens Healthcare GmbH, Erlangen (DE)

(21) Appl. No.: 17/471,721

(22) Filed: Sep. 10, 2021

(30) Foreign Application Priority Data
Sep. 17, 2020 (DE) 10 2020 211 673.3

(51) Int. Cl. G16H 50/30; G16H 40/63; G16H 40/20; A61B 5/00

(52) U.S. Cl. CPC G16H 50/30; A61B 5/747; G16H 40/20; G16H 40/63

(57) ABSTRACT

Methods and systems are for the provision of a stroke classification and for the coordination of emergency services for potential stroke patients. Medical information about a potential stroke patient, including video data of at least the face of the potential stroke patient, is provided. A stroke classification is provided for the potential stroke patient based on the provided medical information. A medical facility for the potential stroke patient can be identified based on the provided stroke classification.

### PRIORITY STATEMENT

The present application hereby claims priority under 35 U.S.C. § 119 to German patent application number DE102020211673.3 filed Sep. 17, 2020, the entire contents of which are hereby incorporated herein by reference.

### FIELD

Example embodiments of the invention generally relate to methods and systems for the provision of a stroke classification and for the coordination of emergency services for potential stroke patients.

### BACKGROUND

Emergency services have the task of providing preliminary medical care, that is to say first aid, for patients in emergency situations and transporting patients to a medical facility such as a hospital with an emergency room as quickly as possible. Emergency service control centers receive incoming emergency calls at a regional or national level and coordinate available emergency services with the aim of ensuring that all patients are transported to appropriate medical care and treatment as quickly as possible. Methods and systems for the coordination of emergency services that direct emergency services to medical facilities for this purpose are already known. These ensure that the patient receives medical care by the fastest route.

It is particularly important in the case of emergencies such as stroke, myocardial infarction and accident that the necessary medical examinations and treatment take place as quickly as possible to avoid any further deterioration in the patient's health and minimize lasting harm. It is necessary for this reason that in the case of stroke, the stroke can be identified as reliably as possible within the framework of a stroke classification and the severity of the stroke can be reliably classified.

Known systems and methods often leave individual emergency service personnel to provide the stroke classification on their own. Misjudgments can have serious consequences on account of the narrow time window available for effective treatment.

It is also important in the case of complex or unclear medical emergencies to provide an accurate provisional diagnosis or initial diagnosis and to transport the patient to a medical facility that is adequately equipped to treat this specific patient. It can happen, for example, that a stroke patient is transported very quickly to a hospital but that the hospital at which they arrive does not have the necessary medical equipment or appropriately trained personnel to provide adequate stroke treatment. The patient may then have to be transported again to an adequately equipped (in terms of medical equipment and personnel) medical facility, possibly after emergency initial examination and treatment at the first hospital. This will significantly increase the time elapsed before the patient receives proper treatment. This can sometimes have grave consequences for the patient, as the longer a stroke remains untreated, the more serious the consequences for the organism of the patient will be. The recovery time for the patient may be extended significantly (for example longer stay at rehabilitation facility) too and the patient may also suffer lasting harm as a result of insufficiently prompt treatment.

Another situation that can occur is that although the patient is transported quickly to a hospital with adequate medical equipment and appropriately trained personnel for the emergency, for example a stroke, the hospital concerned does not have sufficient capacity at the time to care for the patient as quickly as possible or necessary. This can happen if, for example, the hospital at which the patient arrives is already treating a large number of urgent emergency cases and its occupancy rate at that moment is consequently too high to treat the patient as quickly as possible/necessary.

The known methods and systems for the coordination of emergency services, however, do not take account, or do not take sufficient account, of the provisional diagnosis and the availability of medical equipment and/or appropriately trained personnel or of the current occupancy rate of medical facilities when choosing medical facilities, especially in the case of patients who have suffered a stroke (potential stroke patients).

### SUMMARY

At least one embodiment of the present invention is accordingly directed to overcoming, or at least ameliorating, at least one of the disadvantages of the state of the art. At least one embodiment of the present invention to this end creates a method(s) for the provision of a stroke classification and/or for the coordination of emergency services for potential stroke patients and/or a corresponding system(s) for the provision of a stroke classification and/or for the coordination of emergency services for potential stroke patients according to the independent claims. Further embodiments and developments of the present invention are the subject matter of the claims.

According to one embodiment there is created a method for the provision of a stroke classification for a potential stroke patient in a medical information system. This medical information system comprises at least one local device and one central device that has a data link with the at least one local device, and the local device is associated with the potential stroke patient. The method includes:
Receipt of patient identification information from the local device at the central device, which patient identification information uniquely identifies a potential stroke patient in the medical information system;
Querying of patient information data from a database by the central device based on the patient identification information;
Requesting of medical information about the potential stroke patient from the local device by the central device, which medical information includes video data of at least the face of the potential stroke patient;
Receipt at the central device of the medical information from the local device;
Provision of a stroke classification for the potential stroke patient based on the provided medical information and the patient information data.

According to a further embodiment of the present invention, a method for the coordination of emergency services for potential stroke patients includes:
Provision of medical information about a potential stroke patient including video data of at least the face of the potential stroke patient.
Provision of a stroke classification for the potential stroke patient based on the provided medical information.
Identification of a medical facility, in particular an optimal medical facility, for the potential stroke patient based at least on the provided stroke classification.
Provision of location information for the medical facility, in particular an optimal medical facility, identified.

According to a further embodiment of the present invention, a system for the coordination of emergency services for potential stroke patients is able to perform the steps of the method according to the preceding embodiments of the present invention. The system comprises at least one local device and one central device. The system further optionally comprises a second device. The local device is operated by at least one emergency service. The at least one local device is able to perform the step of the provision of medical information and, optionally, the step of the provision of a stroke classification. The central device is operated by an emergency service control center. The central device is able to perform the step of the specification of an optimal medical facility and the step of the provision of location information and, optionally, the step of the provision of a stroke classification. The (optional) second device is operated by a stroke specialist. The (optional) second device is able to perform the step of the provision of a stroke classification.

According to a further embodiment of the present invention, there is provided a system for the provision of a stroke classification for a potential stroke patient. The system comprises the central device able to perform the steps of the methods described herein. The system further optionally comprises at least one of the local devices described herein, with the local device(s) being in each case a mobile terminal or an in-vehicle data processing system of an emergency service vehicle.

According to a further embodiment, the medical facility to which the potential stroke patient should be transported for further examination and treatment is identified based upon (at least) the provided stroke classification. A medical facility is a facility at which the patient can receive medical examination and care, such as a hospital with an emergency room, for example, a physician's practice configured to provide emergency care for patients or another medical center to which emergency services are able to transport patients.

The invention relates in a further embodiment to a computer program product that includes a program and can be loaded directly into a memory of a programmable controller and has program resources, for example libraries and auxiliary functions, to execute a method of at least one embodiment for the provision of a stroke classification or for the coordination of emergency services for potential stroke patients in particular according to the aforementioned embodiments/aspects/developments, when the computer program product is executed.

The invention further relates, in another embodiment, to a computer-readable storage medium in which readable and executable program sections are stored to execute all the steps of a method of at least one embodiment for the provision of a stroke classification or for the coordination of emergency services for potential stroke patients according to the aforementioned embodiments/aspects/developments when the program sections are executed by the controller.

The invention further relates, in another embodiment, to a method for provision of a stroke classification for a potential stroke patient in a medical information system including at least one local device and a central device including a data link with the at least one local device, the local device being associated with a potential stroke patient and the method comprising:
receiving patient identification information from the at least one local device at the central device, the patient identification information uniquely identifying the potential stroke patient in the medical information system;
querying patient information data from a database by the central device based on the patient identification information;
requesting medical information about the potential stroke patient from the at least one local device by the central device, the medical information including video data of at least the face of the potential stroke patient;
receiving at the central device, the medical information from the at least one local device; and
provisioning the stroke classification for the potential stroke patient based on the medical information and the patient information data.

The invention further relates, in another embodiment, to a non-transitory computer-readable storage medium storing readable and executable program sections to execute the method of an embodiment when the program sections are executed by a processing unit.

The invention further relates, in another embodiment, to a medical information system for provision of a stroke classification for a potential stroke patient, comprising:
a central device, configured to perform at least
   receiving patient identification information from the at least one local device, the patient identification information uniquely identifying the potential stroke patient in the medical information system;
   querying patient information data from a database based on the patient identification information;
   requesting medical information about the potential stroke patient from the at least one local device, the medical information including video data of at least the face of the potential stroke patient;
   receiving the medical information from the at least one local device; and
   provisioning the stroke classification for the potential stroke patient based on the medical information and the patient information data.

A non-transitory computer program product storing a program, directly loadable into a memory of a programmable processor of a central device, having program resources, to execute the method of an embodiment when the program is executed.

### BRIEF DESCRIPTION OF THE DRAWINGS

The invention and the technical conditions are explained in detail in the following with reference to the figures. It should be noted that the example embodiments presented are not intended to limit the invention. Specifically, it is also possible, unless explicitly otherwise indicated, to extract sub-aspects of the subject matter explained in the figures and combine them with other elements and knowledge from the present description or figures. It should be noted in particular that the figures and, in particular, the size relationships shown are merely schematic. The same reference signs designate the same objects so that, where applicable, explanatory information from other figures can additionally be applied.

FIG. 1 shows a schematic flow diagram of a method for the coordination of emergency services for potential stroke patients according to an example embodiment.

FIG. 2 shows a schematic view of an example embodiment of the system for the coordination of emergency services and/or for the provision of a stroke classification for stroke patients according to an example embodiment.

FIG. 3 shows a schematic view of an example embodiment of a computer-readable medium.

FIG. 4 shows a schematic view of an example embodiment of a data processing system.

FIG. 5 shows a schematic flow diagram of a method for the provision of a stroke classification for potential stroke patients according to an example embodiment.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. At least one embodiment of the present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

According to one embodiment there is created a method for the provision of a stroke classification for a potential stroke patient in a medical information system. This medical information system comprises at least one local device and one central device that has a data link with the at least one local device, and the local device is associated with the potential stroke patient. The method includes:
Receipt of patient identification information from the local device at the central device, which patient identification information uniquely identifies a potential stroke patient in the medical information system;
Querying of patient information data from a database by the central device based on the patient identification information;
Requesting of medical information about the potential stroke patient from the local device by the central device, which medical information includes video data of at least the face of the potential stroke patient;
Receipt at the central device of the medical information from the local device;
Provision of a stroke classification for the potential stroke patient based on the provided medical information and the patient information data.

This medical information system may, for example, be an electronic health network in which multiple medical facilities are combined and in which at least the potential stroke patient is registered. The central device may include a server unit and, in particular, a web server. The central device may also include a cloud server or a local server. The central device may include one or more real or virtual processing units, such as computers or processors, that are able to process patient information data requested from databases and medical information transmitted by the local device. The central device may additionally have one or more databases to store patient information data or medical information and hold it available for retrieval. The central device may also be able to exchange data with external databases (that is to say databases that, while part of the medical information system, are not part of the central device) in order, for example, to retrieve patient information data stored there.

The central device may have a data link with one or more local device(s) via a communication network. The network may, for example, include the internet or a mobile communication network (such as a wireless network). The central device is able for this purpose to have and manage a data link with multiple local devices, in particular hundreds or thousands of local devices or more.

The local device may in particular be a mobile device, such as a mobile terminal (for example a smartphone or tablet) or a mobile computer, that is installed, for example, in an emergency service vehicle or emergency helicopter. The local device may be associated with the potential stroke patient in the sense that it is situated in the same location as the potential stroke patient. The local device may in particular be such that it can be operated by emergency service personnel treating the potential stroke patient.

According to a further embodiment of the present invention, a method for the coordination of emergency services for potential stroke patients includes:
Provision of medical information about a potential stroke patient including video data of at least the face of the potential stroke patient.
Provision of a stroke classification for the potential stroke patient based on the provided medical information.
Identification of a medical facility, in particular an optimal medical facility, for the potential stroke patient based at least on the provided stroke classification.
Provision of location information for the medical facility, in particular an optimal medical facility, identified.

According to a further embodiment of the present invention, a system for the coordination of emergency services for potential stroke patients is able to perform the steps of the method according to the preceding embodiments of the present invention. The system comprises at least one local device and one central device. The system further optionally comprises a second device. The local device is operated by at least one emergency service. The at least one local device is able to perform the step of the provision of medical information and, optionally, the step of the provision of a stroke classification. The central device is operated by an emergency service control center. The central device is able to perform the step of the specification of an optimal medical facility and the step of the provision of location information and, optionally, the step of the provision of a stroke classification. The (optional) second device is operated by a stroke specialist. The (optional) second device is able to perform the step of the provision of a stroke classification.

According to a further embodiment of the present invention, the central device for the coordination of emergency services is able to perform the step of the provision of a medical facility, in particular an optimal medical facility, and the step of the provision of location information and, optionally, the step of the provision of a stroke classification of the method according to the first embodiment of the present invention.

According to a further embodiment of the present invention, there is provided a system for the provision of a stroke classification for a potential stroke patient. The system comprises the central device able to perform the steps of the methods described herein. The system further optionally comprises at least one of the local devices described herein, with the local device(s) being in each case a mobile terminal or an in-vehicle data processing system of an emergency service vehicle.

It should be noted that some, or even all, of the steps of the method described herein can be performed entirely by one or more correspondingly prepared data processing systems, such as computers. If the method according to the first embodiment of the present invention comprises solely steps that are purely computer-implemented, there is in this case a computer-implemented method for the coordination of emergency services for stroke patients and a data processing system for the coordination of emergency services that encompasses device(s) for performing the steps of the computer-implemented method according to the first embodiment of the present invention.

The term “provision” is used here to denote creation/identification/derivation and/or transmission/forwarding/transfer/display and may involve multiple (sub-)steps.

The term “potential stroke patient” is used here to denote a patient who may be suffering an acute stroke. Correctly diagnosing a stroke is not straightforward and it is sometimes necessary to employ medical imaging procedures to reach a definitive diagnosis, so no more than a provisional diagnosis regarding stroke can be made in the case of emergency deployments of emergency services or first responders. A patient who is suspected of having suffered a stroke is accordingly described herein as a potential stroke patient.

It is assumed herein that emergency services (an emergency services vehicle, emergency physician vehicle, emergency helicopter etc.) has been called or dispatched to an emergency and that the patient is a potential stroke patient. A corresponding emergency call may have been received at an emergency services control center and the emergency services control center may have dispatched emergency services to the emergency based on the information (location of the emergency, nature of the emergency etc.) provided with the emergency call. The emergency service then collected the potential stroke patient with the aim of transporting the patient to an appropriate medical facility as quickly as possible.

According to an embodiment, first of all patient identification information is received. The patient identification information may, for example, be a patient identification number, such as a health insurance scheme number, a patient name etc. The patient identification information has the property, in particular, that it definitively identifies the associated patient in a health network. The central device can in particular take the form of part of such a health network. The patient identification information may in particular be input into the local device by a user of the local device such as a medical worker, for example an emergency physician or paramedic, or even by the actual patient (for example by inputting the patient identification information or by reading in a health card or identity document). The patient identification information can then be transmitted from the local device to the central device.

The central device can then retrieve patient information data for the potential stroke patient, for example from a health database, based on patient identification information. The patient information data may, for example, include an electronic patient file. The patient information data may include personal data such as personal information (for example name, age, gender etc.), medical history (for example pre-existing conditions, prior medical background, medications taken or prescribed etc.), anamnesis (for example anamnesis by emergency service, results of medical tests performed by the emergency service (blood pressure, pulse, ECG and similar) etc.). A precise stroke classification can be derived or autonomously predicted based on the patient information data.

The patient information data contains the medical data about the patient that is available in the central device. The medical data can include both medical image data and non-image data. Image data in this context may be medical image data with two or three spatial dimensions. The image data may also include a time dimension. Medical image data is in particular image data that has been captured using an imaging modality and can in particular depict a part of the patient's body. Imaging modalities here may, for example, be computed tomography systems, magnetic resonance systems, X-ray systems, ultrasound systems and similar. Image data acquired with these or similar modalities is also referred to as radiological image data. The image data may also include longitudinal data, for example in the form of time series or a sequence of images captured at intervals. Non-image data may in particular include longitudinal data that contains one or more medical values for the patient and/or elements from the patient's history of medical conditions. This may be laboratory data, vital signs and/or other measured values or prior examinations relating to the patient. The non-image data may also include demographic details relating to the patient covering factors such as age, gender, lifestyle, risk factors etc., for example. Non-image data may additionally encompass one or more previous findings and/or other assessments (for example by other physicians, possibly the referring physicians). These may be included in the patient information data in the form of one or more structured or unstructured medical findings, for example.

This patient information data may be retrieved from one or more databases. The connected databases can be queried for the patient information data based upon the patient identification information. An electronic identifier such as a patient ID or an access number can be used for this purpose, for example. The patient information data can accordingly be received from one or more of the available databases in which at least parts of the patient information data are stored. The one or more databases may for example be part of medical information systems, such as hospital information systems and/or PACS systems and/or laboratory information systems etc. In particular, one or more such databases may be included in the central device or the central device itself may be part of a medical information system.

The central device may, for the purpose of retrieving the patient information data, have a data query module that is able to query one or more databases (in particular one or more connected databases) for patient information data about the potential stroke patient based on the patient identification information.

Medical information about the stroke patient is then requested in a subsequent step. The medical information includes at least the video data of at least the face of the potential stroke patient. The medical information may additionally include other relevant information about the patient such as, by way of example, personal information (for example name, age, gender etc.), medical history (for example pre-existing conditions, prior medical background, medications taken or prescribed etc.), anamnesis (for example anamnesis by emergency service, results of medical tests performed by the emergency service (blood pressure, pulse, ECG and similar) etc.).

The medical information may be made provided by the local device (for example on request). The medical information may in particular be made available or input into the local device (for example on request) by a user of the local device such as emergency service personnel. The emergency service crew (for example paramedic, emergency physician etc.) can thus record the video data of at least the face of the potential patient with a video camera and transfer it as the medical information using the local device. The emergency service crew can additionally question the potential stroke patient or the potential stroke patient's associates to establish further relevant information about the patient and transfer this further relevant information together with the video data as the medical information.

The local device is preferably operated by the emergency service or, more specifically, the emergency service crew. The local device here may include the video camera with which the video data (image data and, optionally, audio data for the video sequence) of at least the face of the potential stroke patient is captured as (part of the) medical information. The medical information can then be transmitted from the local device to the central device, for example.

The video data shows at least the face of the potential stroke patient. The video data is in particular data from as recent a video sequence as possible or a live video sequence and shows the recent (as recent as possible) or the current condition of the potential stroke patient. The video data may preferably be data from at least one continuous video sequence and additionally include snapshots as well. The video data includes at least image data from a video sequence with, optionally, a predefined minimum running time on which the potential stroke patient can be seen. The video data can additionally include corresponding audio data for the video sequence as well as the image data. The video data of at least the face of the potential stroke patient may also include other regions of the body such as the upper body and arms or the entire body of the potential stroke patient.

The medical information input into the local device can be transmitted from the local device to the central device and received there.

The central device may have an interface module able to exchange data with the local device for the purpose of requesting and receiving the medical information. The interface module may also be able to receive patient identification information from the local device.

The central device then provides a stroke classification based on the medical information received. The stroke classification in some embodiments includes at least one provisional diagnosis of whether or not the case involves a stroke and thus indicates at least whether or not the potential stroke patient is actually to be treated as a stroke patient. Only if stroke can be reliably excluded in this instance is a corresponding stroke classification provided and the potential stroke patient treated as a non-stroke patient. If there remains even the slightest possibility that the potential stroke patient has suffered a stroke, the existence of a stroke can be assumed provisionally and a corresponding stroke classification can be provided so that the potential stroke patient is treated as a stroke patient.

This stroke classification is provided or generated based on the provided medical information, in particular on the video data of at least the face of the potential stroke patient. The video sequence of the face (and optionally of the upper body with arms or the entire body) makes it possible, based on facial expressions typical for stroke (and, optionally, other typical physical manifestations such as paralyzed extremities), to draw a conclusion as to whether/how probable it is that the potential stroke patient is affected by an acute stroke.

The stroke classification can be determined automatically or by involving a stroke expert, with the medical information being provided to the stroke expert if one is involved.

The stroke classification may be provided via the central device having a classification module able to provide the stroke classification based on the patient information data, for example. Alternatively or additionally, the central device may have a communication module able to receive the stroke classification from a second device separate from the central device.

The stroke classification may be determined on the central device or on an expert device operated, for example, by a stroke expert (also referred hereinafter as second device).

The provision of the stroke classification can in particular include transmission of the stroke classification to the local device.

The automated provision of a stroke classification based on current video data that reflects the current condition of the patient and other background information present in the form of patient information data makes it possible to effect a reliable stroke classification. The system is thus enabled to assess the situation correctly and establish the necessary next steps. These steps can then be initiated by the emergency service personnel, for example, or performed by the central device in the form of the automatic assignment of the stroke patient to a medical facility (see above), for example.

Existing system components such as a central device, a database for patient information data or a local device are thus addressed and activated such that they work together in a manner that enables improved and automated stroke classification. The data processing that takes place in the central device is accordingly determined by circumstances outside the data processing system such as, by way of example, the specific patient, the medical information received specifically for the patient or the relevant patient information data. Data is analyzed using technical resources with the objective of deriving a specific result pertinent to the patient (such as a medical diagnosis) by automated device(s) and activating the devices on this basis (for example for the provision or transmission of the stroke classification).

According to a further embodiment, the medical facility to which the potential stroke patient should be transported for further examination and treatment is identified based upon (at least) the provided stroke classification. A medical facility is a facility at which the patient can receive medical examination and care, such as a hospital with an emergency room, for example, a physician's practice configured to provide emergency care for patients or another medical center to which emergency services are able to transport patients.

The medical facility identified may be an optimal medical facility and in particular the medical facility that can provide medical care for the potential stroke patient corresponding to the patient's stroke classification (for example “(probable) stroke case” or “(definite) non-stroke case”). The medical facility should in particular be adequately equipped for the (provisionally) identified emergency, that is to say a potential stroke, meaning that it should have the necessary medical equipment/systems for examination/monitoring (for example imaging system(s), endoscope(s), monitoring device(s) etc.) and treatment (emergency room, operating theater, stents etc.) and should also be the least distant from the emergency service that has collected the emergency patient (potential stroke patient).

This can accordingly be viewed as a type of optimization problem with the object of identifying that medical facility that is least distant from the relevant emergency service and simultaneously has adequate medical equipment to treat the potential stroke patient according to the stroke classification determined. Stored information about the medical equipment of medical facilities and, additionally or alternatively, about the distance of medical facilities from the emergency service may also be used alongside the stroke classification determined to solve this optimization problem. In particular, the shortest distance or journey time for the emergency service to the relevant medical facility can be determined based upon current location information for the emergency service (for example determined using GPS) and the saved location information for each of the medical facilities that are a possibility (that is to say that have adequate medical equipment).

The identification of the (optimal) medical facility can in particular be performed on the central device. The central device may have a coordination module with corresponding capabilities for this purpose.

The location information for the (optimal) medical facility identified is subsequently made available, preferably by the central device. In particular, information about the location of the optimal medical facility identified is sent to the emergency service that has collected the potential stroke patient so that the patient can be provided with optimal medical care as quickly as possible.

Using the provided stroke classification, which is based on the provided medical information, in particular the video data of at least the face of the potential stroke patient, it is possible to identify the (optimal) medical facility for further examination and treatment of the potential stroke patient and provide its location information. The potential stroke patient can thus be given adequate medical care quickly and reliably, which greatly reduces the severity of health consequences and the probability of lasting harm for the potential stroke patient.

According to a development, the methods also include a step, in particular an automatic step, of the identification of specific data that is missing in the patient information data, which missing specific data is relevant in particular for the step of the provision of the stroke classification, with the medical information (MI) comprising the missing specific data identified.

In other words, the missing specific data is requested from the local device by the central device. Missing specific data may in particular include data that is not contained in the patient information data but is relevant for undertaking the stroke classification. This may, for example, be information that is not available in the patient information data because it has not (yet) been entered or is not current, such as, by way of example, demographic data for the patient (for example age, gender), health condition data (for example body mass index, information about medications being taken, blood pressure, information about circulation, oxygen saturation figures etc.).

The requested missing specific data may be provided automatically by the local device and sent to the central device. This can be the case in particular if the local device has an established data link with one or more patient monitoring devices such as, by way of example, an ECG device, a pulse oximeter device etc. Alternatively or additionally, the missing specific data may be entered in the local device by a user of the local device, which then transmits it to the central device.

The central device may, to identify the missing specific data, apply to the patient information data a data analysis algorithm that is able to analyze the patient information data retrieved in relation to the stroke classification to be provided. The data analysis algorithm may, for example, be a rule-based algorithm that queries defined data fields in the patient information data and establishes whether they are populated.

Alternatively or additionally, the data analysis algorithm may be able to determine a predictive confidence value for the stroke classification of the potential stroke patient based on the patient information data retrieved and to identify missing specific data based on the confidence value. The data analysis algorithm may in particular be able to identify missing specific data if the confidence value is lower than a defined limit value. The data analysis algorithm may in particular be able to identify such specific data as missing specific data that if known would raise the confidence value above the defined limit value.

The identification of the missing specific data can be performed by the appropriately configured central device. The central device may have a data analysis module, for example, for this purpose. The data analysis module may, for example, be able to apply the data analysis algorithm to the patient information data.

Identifying missing specific data regarding the stroke classification of the potential stroke patient concerned makes it possible to check proactively whether the information known about the patient is adequate for a stroke classification and, if necessary, to obtain additional data automatically. The stroke classification can thus be improved further, meaning that subsequent processes can be managed with greater precision.

According to a development of the present invention, the method additionally includes the following step:
Provision of a current occupancy rate for at least one medical facility.

In the step involving the identification of an optimal medical facility, the optimal medical facility is additionally identified based on the current occupancy rate provided for the at least one medical facility.

According to a further development, the system additionally optionally includes at least one third device. The (optional) at least one third device is operated by at least one medical facility. The (optional) at least one third device is configured to perform the step of providing a current occupancy rate.

The current occupancy rate indicates what and how much capacity a medical facility has for treating patients. The capacity relates in part to the medical equipment available and in part to the appropriately trained medical personnel available. The current occupancy rate can in particular indicate whether the corresponding medical facility is currently able to provide adequate medical care for a potential stroke patient, that is to say to examine and treat a potential stroke patient.

Information about the current number of patients, the current number of medical personnel, the number and type of medical equipment and the like can be considered to determine the current occupancy rate of a medical facility.

The current occupancy rate can preferably be provided, that is to say determined and, for example, forwarded to the central device, by the at least one third device that is operated by the at least one medical facility.

Additionally considering at least one occupancy rate makes it possible to identify the optimal medical facility for the potential stroke patient with even greater reliability so that the potential stroke patient particularly reliably receives the fastest possible adequate medical care.

According to a development of at least one embodiment of the present invention, the at least one local device is a mobile terminal or an in-vehicle data processing system of an emergency service vehicle. Additionally or alternatively, the second device or the at least one third device is a mobile terminal or a stationary data processing system. Additionally or alternatively, the central device is a stationary data processing system.

The local device and, additionally or alternatively, the second device and, additionally or alternatively, the third device can in particular be mobile terminals such as smartphones, laptops, tablets and similar.

The local device can in particular be an in-vehicle data processing system such as an onboard computer of the emergency service vehicle. The emergency service vehicle may be a land vehicle (for example an ambulance (automobile or motorcycle), emergency physician vehicle (automobile or motorcycle) etc.), an aircraft (for example emergency service helicopter, emergency service airplane etc.) or a watercraft (for example an emergency service boat etc.).

The second device and, additionally or alternatively, the third device and, additionally or alternatively, the central device can in particular be stationary data processing systems such as stationary computers.

The mobile terminals, the in-vehicle data processing system and the stationary data processing systems each include device(s) for performing the corresponding steps of the method according to the first embodiment of the present invention. In particular, they include a data processing unit (CPU), a memory (volatile/non-volatile) (RAM/MEM) and an interface (I/O) to the other or additional devices and, where necessary, a human interface device (HID) (for example a keyboard, a mouse etc.), a display device (MON) (for example a monitor etc.) and a video camera.

Dividing the implementation or performance of the steps across the various devices as described makes it possible to transport the potential stroke patient to the optimal medical facility and thus to provide adequate medical care as quickly as possible in a particularly straightforward and efficient manner.

According to a development of at least one embodiment of the present invention, the step of the provision of a stroke classification includes the following (sub-)steps:
Forwarding of the provided medical information to a trained machine learning algorithm (MLA), in particular a trained neural network (NN).
Autonomous prediction of the stroke classification from the forwarded medical information by the trained MLA, the trained MLA having been trained to predict stroke classifications from medical training information.

The step of the provision of a stroke classification additionally or alternatively includes the following (sub-) steps:
Transmission of the provided medical information to a stroke specialist for the preparation of a provisional diagnosis as to whether the potential stroke patient has a stroke and a corresponding degree of severity; and
Derivation of the stroke classification from a provisional diagnosis prepared based upon the transmitted medical information.

It is to be noted that alternatively, the (sub-)step of the forwarding the provided medical information or the step of the transmission of the provided medical information may be a (sub-)step of the step of the provision of medical information and may be performed on the local device (“push” function, forwarding/transmission is initiated by the local device).

The provided medical information is forwarded for autonomous provision of the stroke classification to the trained MLA or NN so that the trained MLA or NN can use it to predict the stroke classification autonomously.

The MLA or NN may be trained to predict stroke classifications from forwarded medical information based upon “supervised learning” with marked medical training information, “semi-supervised learning” or “unsupervised learning” with unmarked medical training information.

The step of the autonomous prediction of the stroke classification can in particular be performed on the local device or on the central device. The trained MLA or NN to which the provided medical information is forwarded is accordingly implemented on the local or central device. A classification module included in the central device, for example, may be able to host the trained MLA or NN and apply it to the patient information data and the medical information.

The step of the forwarding of the provided medical information may furthermore be performed on the local device or on the central device (“pull” function, forwarding initiated by the local or the central device).

The autonomous prediction or provision of the stroke classification makes it possible to identify the optimal medical facility particularly quickly yet reliably.

The stroke classification is additionally or alternatively transmitted to the stroke specialist. The stroke specialist to whom/which the medical information is additionally or alternatively transmitted is in particular a neurologist or a neurological center. The neurologist or the staff of the neurological center is/are specially trained and instructed in making a (provisional) diagnosis of whether or not the case involves a stroke with reference to the patient's face at least. The provided medical information, in particular the video data of at least the face of the potential stroke patient, is forwarded to the stroke specialist and can be displayed for the latter on a display device (for example a monitor).

The stroke specialist is thus enabled to prepare a provisional diagnosis as to whether the potential stroke patient has a stroke and a corresponding degree of severity based on the transmitted (and displayed) medical information.

The stroke classification is derived from the provisional diagnosis prepared based upon the transmitted medical information. It is thus possible to derive from the provisional diagnosis that the case (definitely) does not involve a stroke, for example, the stroke category of “(definite) non-stroke case”/“(definitely) no stroke”/“0”. Similarly it is possible to derive from the provisional diagnosis that a stroke cannot be (definitely) ruled out, the stroke category of “(probable) stroke case”/“(probable) stroke”/“1”. A stroke category of the stroke classification is thus assigned to the provisional diagnosis. This can be done autonomously by a trained MLA that has been trained accordingly in the classification of natural language or by the actual stroke specialist.

The step of the derivation of the stroke classification can in particular be performed on the second device. The step of the transmission of the provided medical information may furthermore be performed on the second device (“pull” function, transmission initiated by the second device).

It should be noted that the step of the provisional diagnosis by the stroke specialist need not be a part of the present invention. Rather, the present invention may be limited in respect of the provision of the stroke classification to the transmission of the medical information to the stroke specialist and the derivation of the stroke classification from a provisional diagnosis of the stroke specialist.

Enabling a stroke specialist to prepare a provisional diagnosis from the transmitted medical information and using the prepared provisional diagnosis in the derivation of the stroke classification makes it possible to transport the potential stroke patient to a place with adequate medical care particularly reliably and quickly.

According to a development, the medical information further includes a stroke score that is derived from a predefined patient test and, additionally or alternatively, patient information data.

The predefined patient test may in particular be the “Face-Arms-Speech-Time” (FAST) test or an equivalent patient test. The stroke score derived using the predefined patient test may in particular be based on a predefined scale such as the “National Institutes of Health (NIH) Stroke Scale”. The predefined patient test is performed with the potential stroke patient to derive the corresponding stroke score. It is possible based on the stroke score derived to grade the severity of the stroke more precisely or to derive or autonomously predict a more specific stroke classification.

According to a development of at least one embodiment of the present invention, the stroke classification includes at least the categories: “no stroke”; and “stroke”; or at least the categories: “no stroke”; “mild stroke”; and “severe stroke”.

The category “no stroke” or “0” is provided if the stroke patient is (definitely) not suffering a stroke at the time. The categories “stroke”/“1” or “mild stroke”/“1” and “severe stroke”/“2” are provided if the potential stroke patient is (probably) suffering a stroke at the time, the “mild stroke” category being provided if, according to the severity or stroke score, the potential stroke patient is suffering a mild stroke at the time and the “severe stroke” category being provided if, according to the severity or stroke score, the potential stroke patient is suffering a severe stroke at the time. Alternatively, other categories such as from “0” (corresponding to “no stroke”) to “9” (corresponding to “very severe stroke”) may be used.

These categories enable definitive classification of the potential stroke patient and adequate medical care based thereon.

According to a development of at least one embodiment of the present invention, the step of the provision of medical information is performed on a local device operated by an emergency service.

The emergency service or its crew may in particular be able to record the video data, optionally record the stroke score and optionally capture the missing specific data using the local device (for example a smartphone or tablet).

This makes it possible to create/gather and transmit/forward the medical information necessary to provide the stroke classification as quickly as possible. The time elapsed until the potential stroke patient receives adequate medical care is thereby further reduced.

According to a development of at least one embodiment of the present invention, the step of the provision of medical information includes at least one element from the group comprising the (sub-)steps:
Recording or initiating a recording of the video data of at least the face of the potential stroke patient;
Performance or initiation of the performance of the predefined patient test with the potential stroke patient and derivation of the stroke score; and
Determination of the patient information data.

Initiation may in each case involve the transmission of a request from the central device to the local device to record the video data or perform the patient test. Initiation may in each case also involve the transmission of instructions for the recording of the video data or the performance of the patient test.

If one or more of the aforementioned (sub-) steps is/are included in the provision of the medical information and in particular if the (sub-)step(s) concerned is/are performed on the local device, the time that elapses until the potential stroke patient receives adequate medical care is reduced even further.

According to a development of at least one embodiment of the present invention, the step of the provision of a stroke classification is performed either on the local device operated by the emergency service, a second device operated by the stroke specialist or a central device operated by an emergency service control center.

In particular, if the trained MLA is used to provide or predict the stroke classification autonomously, the step of the provision of a stroke classification can be performed autonomously either on the local device or on the central device. This achieves another significant reduction in the time elapsed until the potential stroke patient receives adequate medical care.

If the stroke classification is derived based upon a provisional diagnosis made by the stroke specialist based on the transmitted medical information, the step of the provision of a stroke classification is performed on the second device. This ensures a particularly reliable classification of the stroke patient.

According to a development of at least one embodiment of the present invention, the step of the forwarding of the provided medical information involves the medical information provided by the local device being forwarded by the local device to the trained MLA, which runs on either the local device or the central device. Additionally or alternatively, the step of the transmission of the provided medical information involves the medical information provided by the local device being transmitted from the local device to the second device and the stroke classification being derived, in the step of the derivation of the stroke classification, by the second device.

This ensures a reliable and quick flow of information between the devices irrespective of whether the stroke classification is provided autonomously on the local or the central device or is provided on the second device using the provisional diagnosis made by the stroke specialist. The potential stroke patient can thus be transported to adequate medical care as quickly as possible.

According to a development of at least one embodiment of the present invention, the step of the provision of medical information additionally includes the following step:
Establishment of a bidirectional communication link between the local device and the second device, optionally by the central device.

The video data recorded of at least the face of the potential stroke patient is preferably transmitted as an essentially instantaneous transmission from the local device to the second device via the bidirectional communication link. Optionally, the predefined patient test is performed by the stroke specialist with the potential stroke patient via the bidirectional communication link.

The bidirectional communication link makes it possible for video data encompassing image data and audio data to be exchanged in both directions in real time without delay between the local device and the second device. It is sufficient in this connection if image data is transmitted only from the local device to the second device and audio data is transmitted in both directions. Instantaneous or without delay is understood in the present context to mean that the only delays affecting transmission are possible technical delays and the video data is not stored temporarily for subsequent transmission.

The bidirectional communication link enables the stroke specialist to examine the potential stroke patient (in particular the potential stroke patient's face) live in a video communication session or video conference with the potential stroke patient to make the diagnosis. The stroke specialist is able to present questions and instructions to the potential stroke patient live and can thus optionally perform the predefined patient test to determine the stroke score live with the potential stroke patient as well.

This ensures that as reliable a stroke classification as possible is provided as quickly as possible and that the optimal medical facility to which to transport the potential stroke patient can be identified on this basis.

According to a development of at least one embodiment of the present invention, the step of the provision of a stroke classification also includes the following (sub-)step:
Transmission of the provided stroke classification to the central device.

The steps of the identification of an optimal medical facility and the provision of location information are performed on the central device. The step of the provision of location information includes the following (sub-)step:
Transmission of the provided location information from the central device to the local device.

This configuration ensures that all the necessary information is available at the relevant devices as quickly as possible and the emergency service can reach the optimal medical facility as quickly as possible.

According to a development of at least one embodiment of the present invention, the step of the provision of a current occupancy rate is performed on at least one third device operated by at least one medical facility. The method additionally includes the following step:
Transmission of the provided current occupancy rate from the at least one third device to the central device.

The medical facilities share their current occupancy rate with the emergency service control center continuously so that the relevant current occupancy rate can be factored in when identifying the optimal medical facility.

The current occupancy rate provided directly by the medical facilities makes it possible to identify the optimal medical facility to receive the potential stroke patient with even greater certainty.

According to a development of the present invention, the method additionally includes the step:
Provision of a patient drop notification, optionally by the central device, including the provided stroke classification and optionally at least some of the provided medical information about the potential stroke patient, which optionally was transmitted from the local device to the central device, and optionally an estimated time of arrival.

The step of the provision of a patient drop notification includes the (sub-)step:
Transmission (S61) of the provided patient drop notification (CN), optionally by the central device (4), to the third device (3) operated by the optimal medical facility identified.

According to a further development, the central device is able to perform the step of the provision of a patient drop notification.

The estimated time of arrival is the time until or clock time at which the emergency service is expected to arrive with the potential stroke patient at the optimal medical facility identified. The estimated time of arrival may optionally be updated continuously and transmitted to the optimal medical facility. The estimated time of arrival can be determined, preferably by the central device or alternatively by the local device, based upon the current location information for the emergency service (for example as determined by GPS) and the saved location information for the optimal medical facility.

The patient drop notification provided, which is transmitted to the optimal medical facility identified, enables medical personnel at the optimal medical facility to respond promptly to the potential stroke patient and, where appropriate, to make necessary preparations even before the emergency service arrives.

According to a development, at least one element of the group comprising: the provided medical information; the provided stroke classification; the provided location information; the provided current occupancy rate; and the provided patient drop notification is transmitted or forwarded in encrypted form.

This encryption is preferably realized in accordance with regionally or nationally specified standards or guidelines and particularly preferably in accordance with standards that are required by the “Health Insurance Portability and Accountability Act” (HIPAA).

According to a development, the methods can also include the following steps:
Provision in each case of patient information data for multiple comparison patients, each comparison patient being associated with a known stroke classification; and
Identification of one or more reference patient(s) from multiple comparison patients based on similarity indicators, it being the case that a similarity indicator is based on a similarity between the patient information data for the potential stroke patient and the patient information data for the comparison patients.

The stroke classification is in this case additionally provided based upon the known stroke classifications of the reference patients in the step of the provision of the stroke classification.

It is envisaged, in other words, to improve the stroke classification with reference to the patient information data for the potential stroke patient by seeking to process the case automatically in accordance with similar cases for which a stroke classification already effected is (already) known and, optionally, has been verified. This approach is based on the notion that knowledge gained from cases of a similar nature may potentially be relevant for the present case. It is intended for this purpose to identify from a quantity of comparison patients reference patients who exhibit a certain similarity with the potential stroke patient. This is done by comparing the patient information data for the potential stroke patient with the patient information data for each of the comparison patients. The patient information data for the comparison patients may have a similar structure and content to the patient information data for the potential stroke patient. The patient information data for the comparison patients may be stored in one or more databases that are simultaneously part of a (the) medical information system. In particular the

All the available patient information data for the comparison patients can be examined for its similarity to the patient information data for the potential stroke patient to identify the reference patients. A similarity indicator that is based on a similarity between the patient information data for the relevant comparison patient and the potential stroke patient and that in particular specifies or quantifies a similarity may be determined for each of the comparison patients. A similarity indicator may for example be a numerical value or score. The similarity indicators may, for example, be determined based on the application of a similarity metric that outputs a similarity indicator based on the input variables, that is to say the patient information data. The similarity metric in this case can in particular be implemented in a data processing algorithm hosted, by way of example, in the classification module of the central device. Reference patients are in particular those comparison patients who exhibit a certain similarity with the potential stroke patient based upon the respective patient information data. In other words, reference patients can in particular be those comparison patients whose similarities in respect of the patient information data exceed a predefined/specified or specifiable threshold.

Each comparison patient is associated with at least one stroke classification, so the automatic search for similar patients supplies a selection of stroke classifications that are possibly relevant for the potential stroke patient.

According to a development, the identification of one or more reference patients includes the steps:
Extraction of a data descriptor from the patient information data for the potential stroke patient;
Receipt of a corresponding data descriptor for each of the comparison patients;
Determination of a similarity indicator for each comparison patient, a similarity indicator being based in each case on a similarity between the data descriptor and a corresponding data descriptor; and
Identification of the one or more reference patient(s) based on the similarity indicators determined.

The data descriptor may have one or more attributes extracted from or calculated from the patient information data. The expression “attribute signature” can be another name for data descriptor. The data descriptor can in particular characterize the patient information data. The attributes of the data descriptor may be combined into an attribute vector. The data descriptor can in particular have such an attribute vector. Attributes extracted from image data may be morphological and/or structural attributes and/or attributes relating to a texture and/or a pattern. Attributes extracted from non-image data may be attributes relating to a finding, a medical report, a measured value, an item of demographic information etc. The classification module of the central device can in particular be configured to determine similarity indicators based on the data descriptor or to host a corresponding data processing algorithm.

The determination of the similarity indicators may include the extraction or receipt in each case of a corresponding data descriptor from the patient information data for the comparison patient. The determination of the similarity indicators may also include a comparison of the respective corresponding data descriptors with the data descriptor. The comparison step can in particular be based on the determination of a distance separating the respective data descriptors in the attribute space, the calculation of a cosine similarity of the data descriptors and/or the calculation of a weighted sum of the difference or similarity of individual attributes of the data descriptor. The comparison patients identified as reference patients can in particular be those comparison patients whose associated similarity indicator exceeds a specified or specifiable threshold.

The use of data descriptors makes it possible to define easily implemented and readily transferable parameters for comparing different patient information data. The attributes contained in the attribute signatures can in addition be based on higher-level observables, which higher-level observables are derived from the data records and often characterize the properties of the data records better than the underlying data itself.

According to one embodiment, the identification of the one or more reference patients includes the application of a trained function in each case to the patient information data of the potential stroke patient and the comparison patient, which trained function is able to determine a similarity indicator between patient information data or to extract data descriptors from patient information data and determine a similarity indicator between patient information data items based upon the extracted data descriptors.

A trained function generally maps input data to output data. This output data can in particular be dependent on one or more parameters of the trained function. The one or more parameters of the trained function may be determined and/or adjusted by training. The determination and/or adjustment of the one parameter or multiple parameters of the trained function can in particular be based on a pair of training input data items and associated training output data items, it being the case that the trained function is applied to the training input data to generate training output data. The determination and/or adjustment can in particular be based on a comparison of the training mapping data and the training output data. A trainable function, that is to say a function with parameters yet to be adjusted, is generally also referred to as a trained function. According to example embodiments of the invention, such a trained function can take the form of a neural network or a convolutional neuronal network.

The invention relates in a further embodiment to a computer program product that includes a program and can be loaded directly into a memory of a programmable controller and has program resources, for example libraries and auxiliary functions, to execute a method of at least one embodiment for the provision of a stroke classification or for the coordination of emergency services for potential stroke patients in particular according to the aforementioned embodiments/aspects/developments, when the computer program product is executed.

The invention further relates, in another embodiment, to a computer-readable storage medium in which readable and executable program sections are stored to execute all the steps of a method of at least one embodiment for the provision of a stroke classification or for the coordination of emergency services for potential stroke patients according to the aforementioned embodiments/aspects/developments when the program sections are executed by the controller.

The computer program products may in this case include software with a source code that still has to be compiled and linked or that only has to be interpreted, or an executable software code that for execution has only to be loaded into the processing unit. The computer program products make it possible to execute the methods quickly and robustly in a manner that allows them to be repeated in identical form. The computer program products are configured such that they are able to execute method steps according to the invention with the processing unit. The processing unit in this case must satisfy each of the necessary conditions such as, by way of example, appropriate working memory, an appropriate processor, an appropriate graphics card or an appropriate logic unit so that the respective method steps can be executed efficiently.

The computer program products are stored, by way of example, on a computer-readable storage medium or filed on a network or server from where they can be loaded into the processor of the relevant processing unit, which processor may be directly connected with the processing unit or realized as part of the processing unit. Control information of the computer program products may in addition be stored on a computer-readable storage medium. The control information for the computer-readable storage medium may be in such a form that it performs a method according to the invention when the data storage medium is used in a processing unit. Examples of computer-readable storage media are a DVD, a magnet tape or a USB stick on which is stored electronically readable control information, in particular software. All of the embodiments/aspects of the methods according to the invention previously described can be performed when this control information is read from the data storage medium and saved in a processing unit. The invention can thus also be based on the computer-readable medium and/or the computer-readable storage medium. The advantages of the proposed computer program products and/or the associated computer-readable media essentially correspond to the advantages of the proposed methods.

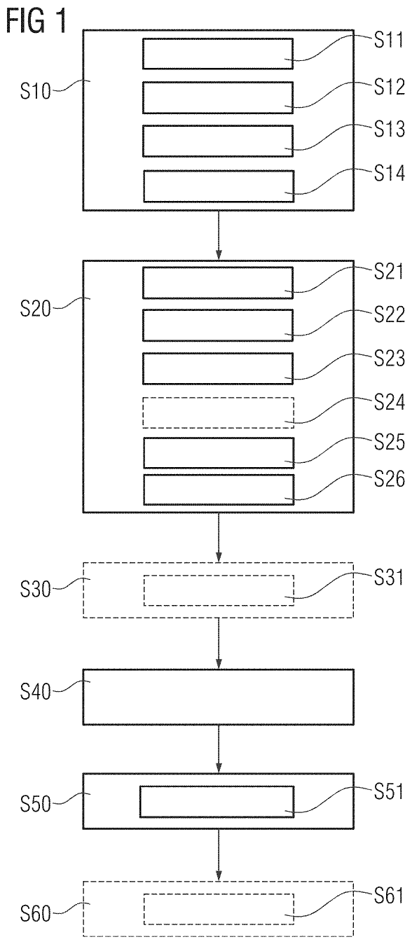

FIG. 1 presents an example embodiment of the method for the coordination of emergency services for potential stroke patients according to the first embodiment of the present invention. The method includes the steps provision S10 of medical information, provision S20 of a stroke classification, identification S40 of an optimal medical facility and provision S50 of location information plus, optionally, provision S30 of a current occupancy rate and, optionally, provision S60 of a patient drop notification.

The medical information provided in the provision S10 of medical information step is medical information pertaining to a potential stroke patient. The medical information includes video data of the face and upper body with arms of the potential stroke patient plus a stroke score and patient information data.

The provision S10 of medical information step accordingly includes the sub-steps establishment S11 of a bidirectional communication link, recording S12 of the video data, performance S13 of the predefined patient test and determination S14 of the patient information data.

The bidirectional communication link in the establishment S11 of a bidirectional communication link sub-step is established between a local device and a second device. The bidirectional communication link may for example be established via a wireless network or another communication network. It is possible using the bidirectional communication link for image data and audio data for the video data of the face and the upper body with arms of the potential stroke patient to be transmitted from the local device to the second device and for image data and audio data for video data of a stroke patient to be transmitted from the second device to the local device.

The video data in the recording S12 of the video data sub-step is recorded of at least the face and the upper body with arms of the potential stroke patient. The video data recorded is transmitted live from the local device to the second device via the bidirectional communication link established. The video data of the stroke specialist is additionally recorded and transmitted live from the second device to the local device via the bidirectional communication link. The stroke specialist is thus able to maintain live video communication with the potential stroke patient.

In the performance S13 of the predefined patient test sub-step, the FACE test is performed with the potential stroke patient by the stroke specialist using the live video communication session and the stroke score is derived based upon the FACE test.

In the determination S14 of the patient information data sub-step, the patient information data including personal data such as personal information (for example name, age, gender etc.), medical history (for example pre-existing conditions, prior medical background, medications taken or prescribed etc.), anamnesis (for example anamnesis by emergency service, results of medical tests performed by the emergency service (blood pressure, pulse, ECG and similar) etc.) is determined.

In the provision S20 of a stroke classification step, the stroke classification based on the provided medical information is provided. The stroke classification includes the categories “no stroke” (“0”), “mild stroke” (“1”) and “severe stroke” (“2”).

The provision S20 of a stroke classification step to this end includes the sub-steps forwarding S21 of the provided medical information and autonomous prediction S22 of the stroke classification or the sub-steps transmission S23 of the provided medical information and derivation S25 of the stroke classification plus the step transmission S26 of the provided stroke classification.

The provided medical information is forwarded in the forwarding S21 of the provided medical information sub-step from the local device to a trained neural network (NN), for example via cable-bound communication or a wireless network or other communication network.

The stroke classification in the autonomous prediction S22 of the stroke classification sub-step is predicted autonomously by the trained NN from the forwarded medical information. The trained NN has been trained to predict stroke classifications autonomously using medical training information.

The transmission S23 of the provided medical information sub-step and the derivation S25 of the stroke classification sub-step may be performed in parallel with/in addition to or alternatively to the preceding sub-steps.

The provided medical information in the transmission S23 of the provided medical information sub-step is transmitted to a stroke specialist (for example a neurologist) via the provided medical information being transmitted from the local device to the second device, for example via a wireless network or other communication network. The stroke specialist is able to make a provisional diagnosis as to whether the potential stroke patient has a stroke and specify a corresponding degree of severity (intimated by sub-step S24) based upon the transmitted medical information, in particular the live video data of the face and the upper body with arms of the potential stroke patient.

The stroke classification in the derivation S25 of the stroke classification sub-step is derived based upon the provisional diagnosis generated using the transmitted medical information.

The provided stroke classification in the transmission S26 of the provided stroke classification sub-step is forwarded or transmitted to a central device by the trained NN or by the second device.

The current occupancy rate for all available medical facilities is provided in the optional provision S30 of a current occupancy rate step. The available medical facilities make their respective current occupancy rates available continuously via a relevant third device for this purpose. The occupancy rate indicates how much medical care capacity (medical equipment and personnel) the medical facility concerned currently has available.

The optional provision S30 of a current occupancy rate step to this end includes the sub-step transmission S31 of the provided current occupancy rate.

In the transmission S31 of the provided current occupancy rate sub-step, the provided current occupancy rates of all available medical facilities are transmitted from their respective third device to the central device.

In the identification S40 of a medical facility step, a (the optimal) medical facility for the potential stroke patient is identified from all the available medical facilities based at least on the provided stroke classification and, optionally, the provided occupancy rates of the available medical facilities. Stored information about the medical equipment of all the available medical facilities is also considered in this context so that medical facilities that are not equipped to treat a stroke patient are not identified as the optimal medical facility for potential stroke patients with a provided stroke classification other than “no stroke” (“0”). The step of the identification S40 of an optimal medical facility is performed on the central device.

In the provision S50 of location information step, the location information for the optimal medical facility identified is provided by the central device. Location information for the respective available medical facilities is stored in the central device for this purpose.

The provision S50 of location information step also includes the sub-step transmission S51 of the provided location information.

The provided location information in the transmission S51 of the provided location information sub-step is transmitted from the central device to the local device.

In the optional provision S60 of a patient drop notification step, the patient drop notification including the provided stroke classification, the provided medical information about the potential stroke patient transmitted from the local device to the central device and an estimated time of arrival is provided by the central device. The estimated time of arrival is the time until or clock time at which the emergency service is expected to arrive with the potential stroke patient at the optimal medical facility identified.

The provision S60 of a patient drop notification step includes the sub-step transmission S61 of the provided patient drop notification.

The provided patient drop notification in the transmission S61 of the provided patient drop notification sub-step is transmitted from the central device to the third device of the identified optimal medical facility.

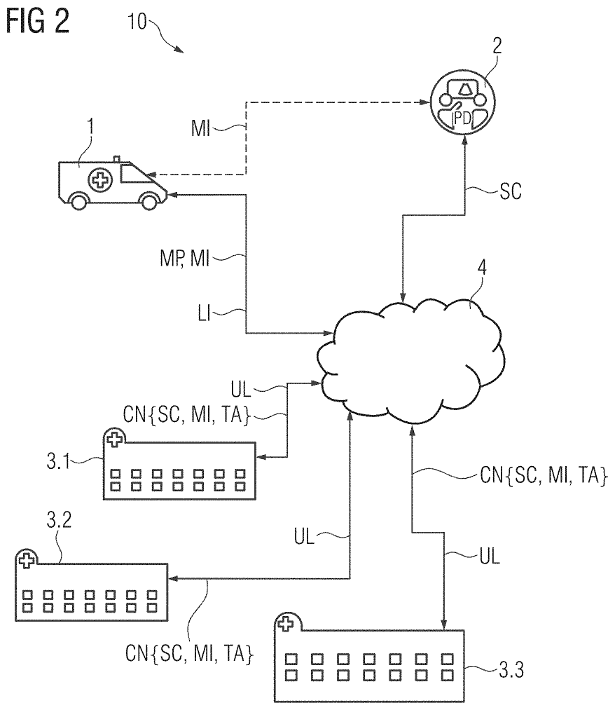

FIG. 2 presents an example embodiment of a medical information system 10 for the provision of a stroke classification or for the coordination of emergency services for stroke patients in schematic form. The system includes multiple local devices 1 (only one of which is shown here), a second device 2, multiple third devices 3 and a central device 4.

The local devices 1 are mobile terminals that are each allocated to an emergency service (in this case an ambulance) and are operated by the emergency service or the emergency service crew (paramedic, emergency physician). The local devices 1 are linked with the central device 4 for communication purposes, for example via a wireless network.

The second device 2 is a stationary data processing system operated by a stroke specialist. The second device is linked with the central device 4 for communication purposes, for example via the internet.

The third devices 3.1-3.3 are stationary data processing systems operated by available medical facilities or their personnel. The third devices 3.1-3.3 are linked with the central device 4 for communication purposes, for example via the internet.

The central device 4 may be operated, by way of example, by an emergency service control center or its personnel.

If an emergency service is called to an emergency and if the emergency turns out to involve a potential stroke patient, step S10 and its sub-steps S11 through S14 can be performed on the local device 1 of the emergency service. The medical information MI about the stroke patient is provided in this case via the establishment of a bidirectional communication link (dashed double-headed arrow) between the local device 1 and the second device 2, the recording of video data of the face and upper body of the stroke patient and the determination of the patient information data and any missing specific data. The local device 1 in addition continuously transmits current location information MP for the emergency service to the central device 4.

Step S20 and its sub-steps S23 through S26 can be performed on the central device 4, with the optional partial participation of a second device 2, based on the bidirectional link established between the local device 1 and the second device 2. The medical information may in this case also be transmitted from the local device 1 to the second device 2 and the stroke classification SC can be derived based upon a provisional diagnosis PD as to whether the potential stroke patient has a stroke via the assignment by the stroke specialist of one of the three categories “no stroke”, “mild stroke” and “severe stroke” to the stroke specialist's provisional diagnosis using the second device. The stroke specialist can additionally perform the FACE test with the patient live via the bidirectional communication link. The transmitted medical information here enables the stroke specialist to make the provisional diagnosis PD. The derived stroke classification SC is then transmitted to the central device 4.

In parallel with the above, step S30 and its sub-step S31 can be performed continuously by the third devices and current occupancy rates (UL) for the available medical facilities can be transmitted from the third devices 3 to the central device 4.

The central device 4 can in addition perform step S40 and then identifies the optimal medical facility for the further medical care of the potential stroke patient based on the transmitted stroke classification SC, the transmitted occupancy rates UL and the transmitted current location information MP plus stored information about the medical equipment of the available medical facilities and the stored location information LI for the available medical facilities.

The central device 4 can in addition perform step S50 and its sub-step S51 by determining location information LI for the identified optimal medical facility and transmitting this location information LI to the local device.

The central device 4 can also perform step S60 and its sub-step S61 by generating the patient drop notification CN and transmitting it to the third device 3 of the identified optimal medical facility with the provided stroke classification SC, the provided medical information MI about the potential stroke patient transmitted from the local device 1 to the central device 4 and the estimated time of arrival TA.

The central device 4 can in addition perform the steps S100, S200, S300 and S400, S500 (see FIG. 5) to derive a stroke classification.

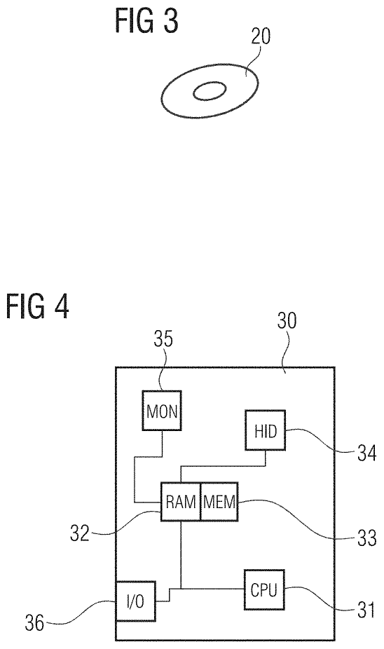

FIG. 3 presents an embodiment of a computer-readable medium 20 in schematic form.

Here, as an example, a computer-readable storage disk 20 such as a compact disc (CD), digital video disc (DVD), high definition DVD (HD DVD) or Blu-ray disc (BD) has stored on it a computer program that includes instructions that, when executed by a data processing system (computer), cause the data processing system to execute step S20 with sub-steps S21 and S22, step S40, step S50 with sub-step S51 and step S60 with sub-step S61.

The computer-readable medium can though also be a data memory such as a magnetic memory (for example a magnetic-core memory, magnetic tape, magnetic card, magnetic strip, magnetic bubble memory, drum module, hard disk, diskette or removable medium), an optical memory (for example a holographic memory, optical tape, tesafilm, laser disc, Phasewriter (Phasewriter Dual, PD) or Ultra Density Optical disk (UDO)), a magneto-optical memory (for example a MiniDisc or magneto-optical disk (MO)), a volatile semiconductor/solid-state memory (for example random access memory (RAM), dynamic RAM (DRAM) or static RAM (SRAM)) or a non-volatile semiconductor/solid-state memory (for example read only memory (ROM), programmable ROM (PROM), electrically erasable EPROM (EEPROM), flash EEPROM (for example a USB stick), ferroelectric RAM (FRAM), magnetoresistive RAM (MRAM) or phase-change RAM).

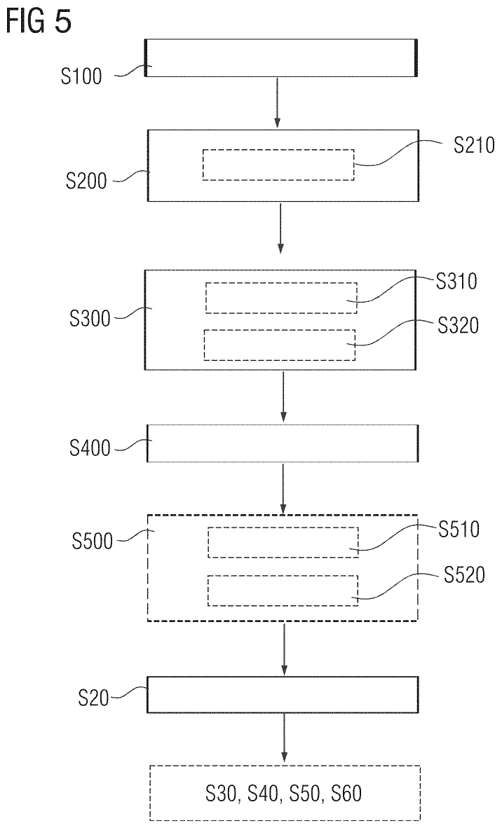

FIG. 4 presents an example embodiment of a data processing system 30 in schematic form. The data processing system 30, which may be the central device 4, can perform step S20 with sub-steps S21 and S22, step S40, step S50 with sub-step S51 and step S60 with sub-step S61 plus steps S100, S200, S300, S400, S500 with the relevant sub-steps.

The data processing system 30 can be a personal computer (PC), a laptop, a tablet, a server, a distributed system (for example a cloud system) or similar. The data processing system 30 includes a central processing unit (CPU) 31, memory including random access memory (RAM) 32 and non-volatile memory (MEM, for example a hard disk) 33, a human interface device (HID, for example a keyboard, mouse, touchscreen etc.) 34, an output device (MON, for example a monitor, printer, loudspeaker etc.) 35 and an interface (input/output, I/O, for example USB, Bluetooth, WLAN etc.) 36 for receiving and sending data. The CPU 31, the RAM 32, the HID 34, the MON 35 and the I/O 36 are linked for communication purposes via a data bus. The RAM 32 and the MEM 33 are linked for communication purposes via a different data bus.

The computer program stored on the computer-readable medium 20 can be stored in the MEM 33 and loaded into the RAM 32 from here or the computer-readable medium 20. The CPU 31 performs step S20 with sub-steps S21 and S22, step S40, step S50 with sub-step S51 and step S60 with sub-step S61 according to the computer program. Execution can be initialized and controlled by a user (emergency service control center personnel) via the HID 34. The status and the result of the computer program executed can be displayed to the user by the MON 35 or forwarded via the I/O. The result of the computer program executed can be stored permanently on the non-volatile MEM 33 or a different computer-readable medium.

The CPU and the RAM 32 can in particular include multiple CPUs 31 and multiple RAMs 32, for example in a computer cluster or a cloud system, for executing the computer program. The HID 34 and the MON 35 for controlling the execution of the computer program can be included in another data processing system such as a terminal that is linked with the data processing system 30 (for example a cloud system) for communication purposes.

FIG. 5 shows a schematic flow diagram of a method for the provision of a stroke classification for potential stroke patients according to an example embodiment. The sequence of the method steps is constrained neither by the depicted sequence nor by the chosen numbering. It is thus possible to change the sequence of the steps where applicable and to omit individual steps. It is also possible for the execution of one or more steps, in particular a sequence of steps, and optionally the entire method to be repeated. Identical reference signs to those described in relation to FIG. 1 designate identical method steps.

In a first step S100, an item of patient identification information from the local device 1 is received at the central device 4. The patient identification information may for example be provided by the local device 1 and transmitted to the central device 4 via a communication network that forms a data link between the central device 4 and the local device 1.

In step S200, the central device 4 queries patient information data for the potential stroke patient from a database with which the central device 4 (but not the local device 1) has a data link. A search query for corresponding patient information data based on the patient identification information, for example, can be formulated for this purpose. In an optional sub-step S210, the patient information data is analyzed by the central device 4 to ascertain whether the patient information data is suitable for the subsequent determination of a stroke classification as it is, that is to say, in other words, whether it contains all the information relevant for this purpose. If the patient information data is not suitable as it is, specific data this is missing in the patient information data can be identified in step S210.

In step S300, the central device 4 requests medical information MI from the local device 1. This may, for example, be video data of a face of the potential stroke patient. Step S300 can accordingly include an optional sub-step S310 of the initiation S310 of a recording S12 of the video data of at least the face of the potential stroke patient and an optional sub-step S320 of the initiation S320 of a performance S13 of the predefined patient test with the potential stroke patient and derivation of the stroke score. The sub-steps S310 and S320 can each include the transmission of electronic instructions for the recording of the video data or for the performance of the predefined patient test for the emergency service personnel or the patient. If missing specific data is identified in step S210, this missing specific data can also be requested as part of the medical information MI in the course of step S300.

In step S400, the medical information MI is received by the central device 4. The local device 1 provides this medical information and transmits it to the central device 4 via the communication network.

In an optional step S500, the central device 4 performs a similarity analysis with reference to the patient information data for the potential stroke patient. The aim of this step is to identify reference patients who show a similar clinical course based on the patient information data. If there is a verified stroke classification available for such patients, this information can be used to improve the provision of the stroke classification in step S20. In an optional sub-step S510, patient information data of multiple comparison patients is to this end first retrieved, each of the comparison patients being associated with a known stroke classification. In an optional sub-step S520, one or more reference patients are then identified from multiple comparison patients based on similarity indicators, it being the case that a similarity indicator is based on a similarity between the patient information data for the potential stroke patient and the patient information data for one of the comparison patients. The stroke classification can then optionally be determined based upon the known stroke classification for the reference patients as well in step S20.

Step S20 can optionally be followed by steps S30, S40, S50, S60 described above, each with one or more of the sub-steps described in this connection.

Although specific embodiments have been illustrated and described here, it will be apparent to the person skilled in the art that there are a multiplicity of alternatives and/or equivalent implementations. It should be acknowledged that the example embodiments or embodiment variants are just examples and are not intended to limit the scope, applicability or configuration in any way. The above summary and detailed description will rather provide the person skilled in the art with sufficient guidance to implement at least one preferred embodiment, it being understood that different changes in the function and arrangement of the elements that are described in an example embodiment do not go beyond the scope of application presented in the attached claims and their legal equivalents. The present application is generally intended to cover all adaptations or variations of the specific embodiments discussed herein.

Various attributes have been combined in one or more examples in the preceding detailed description in order to keep the disclosure concise. It is clear that the above description is intended to be illustrative rather than restrictive and to cover all alternatives, modifications and equivalents that may be included within the framework of the invention. Many other examples will become apparent to the person skilled in the art on studying the above disclosure.

A specific nomenclature is used and has been used in the preceding disclosure to facilitate a comprehensive understanding of the invention. It will, however, be apparent to the person skilled in the art, in light of the specification contained therein, that the specific details are not essential to apply the invention. The preceding descriptions of specific embodiments of the present invention are thus presented for the purposes of illustration and description. They are not intended to be exhaustive or to limit the invention to the exact embodiments disclosed above; obviously there are many modifications and variations possible in respect of the aforementioned teachings. The embodiments have been selected and described in order to elucidate the principles of the invention and its practical applications as effectively as possible and thus to enable other specialists to apply the invention and different embodiments with different modifications, as appears appropriate for the relevant use, as effectively as possible. Throughout the specification, the terms “including” and “in which” are used in a sense equivalent to the terms “encompassing” and “it being the case that”. The terms “first”, “second”, “third” etc. are used merely as a designation and are not intended to impose numerical requirements regarding the objects or to specify a particular order. The conjunction “or” is used in the present description and the claims in the inclusive (“and/or”) rather than the exclusive (“either . . . or”) sense.

The following items also form part of the disclosure:

1. A method for the coordination of emergency services for potential stroke patients encompassing the steps:
provision of medical information about a potential stroke patient including video data of at least the face of the potential stroke patient;
provision of a stroke classification for the potential stroke patient based on the provided medical information;
identification of an optimal medical facility for the potential stroke patient based at least on the provided stroke classification; and
provision of location information for the optimal medical facility identified.
   2. The method as claimed in claim 1, additionally encompassing the step:
provision of a current occupancy rate for at least one medical facility, wherein in the step involving the identification of an optimal medical facility, the optimal medical facility is additionally identified based on the current occupancy rate provided for the at least one medical facility.
   3. The method as claimed in one of the preceding items, wherein
   the step of the provision of a stroke classification encompasses the steps:
forwarding of the provided medical information to a trained machine learning algorithm, MLA, in particular a trained neural network, NN; and
autonomous prediction of the stroke classification from the forwarded medical information by the trained MLA, wherein the trained MLA has been trained to predict stroke classifications from medical training information;
   or the steps:
transmission of the provided medical information to a stroke specialist for the preparation of a provisional diagnosis as to whether the potential stroke patient has a stroke; and
derivation of the stroke classification from a provisional diagnosis prepared based upon the transmitted medical information.
   4. The method as claimed in one of the preceding items, wherein the medical information further includes at least a stroke score that is derived from a predefined patient test and, additionally or alternatively, patient information data.
   5. The method as claimed in 4,
   the step of the provision of medical information including at least one element from the group comprising the steps:
recording of the video data of at least the face of the potential stroke patient;
performance of the predefined patient test with the potential stroke patient and derivation of the stroke score; and
determination of the patient information data.
   6. The method as claimed in one of the preceding items, the stroke classification including at least the categories:
“no stroke”; and “stroke”;
   or at least the categories:
“no stroke”; “mild stroke”; and “severe stroke”.
   7. The method as claimed in one of the preceding items, wherein the step of the provision of medical information (MI) is performed on a local device operated by an emergency service.
   8. The method as claimed in one of the preceding items, wherein the step of the provision of a stroke classification is performed either on the local device operated by the emergency service, a second device operated by the stroke specialist or a central device operated by an emergency service control center.
   9. The method as claimed in 8, wherein the step of the forwarding of the provided medical information involves the medical information provided by the local device being forwarded by the first device to a trained machine learning algorithm, MLA, in particular a trained neural network, NN, which runs on either the first device or the central device and has been trained to predict stroke classifications from medical training information, or
   wherein the step of the transmission of the provided medical information involves the medical information provided by the local device being transmitted from the local device to the second device and the stroke classification being derived, in the step of the derivation of the stroke classification, by the second device.
   10. The method as claimed in 8,
   the step of the provision of medical information additionally including the step:
establishment of a bidirectional communication link between the local device and the second device, optionally by the central device,
   wherein the video data recorded of at least the face of the potential stroke patient is transmitted as an instantaneous transmission from the local device to the second device via the bidirectional communication link and, optionally, wherein the predefined patient test is performed by the stroke specialist with the potential stroke patient via the bidirectional communication link.

11. The method as claimed in one of items 8 to 10,

the step of the provision of a stroke classification additionally including the step:
transmission of the provided stroke classification to the central device,
   wherein the steps of the identification of an optimal medical facility and the provision of location information are performed on the central device and the step of the provision of location information including the step:
transmission of the provided location information from the central device to the local device.
   12. The method as claimed in 2 in combination with one of items 8 to 11,
   wherein the step of the provision of a current occupancy rate is performed on at least one third device operated by at least one medical facility,
   the method additionally includes the step:
transmission of the provided current occupancy rate from the at least one third device to the central device.
   13. The method as claimed in one of the preceding claims, additionally including the step:
provision of a patient drop notification, optionally by the central device, including the provided stroke classification and optionally at least some of the provided medical information about the potential stroke patient, which optionally was transmitted from the local device to the central device, and optionally an estimated time of arrival, the step of the provision of a patient drop notification step including the step:
   transmission of the provided patient drop notification, optionally by the central device, to a third device operated by the optimal medical facility identified.
      14. The method as claimed in one of the preceding claims, wherein at least one element of the group comprising: the provided medical information; the provided stroke classification; the provided location information; the provided current occupancy rate; and the provided patient drop notification is transmitted or forwarded in encrypted form.
      15. A system for the coordination of emergency services for potential stroke patients that is able to perform the steps of the method as claimed in one of the preceding claims, including:
      at least one local device operated by at least one emergency service and able to perform the step of the provision of medical information and, optionally, the step of the provision of a stroke classification; and
      a central device operated by an emergency service control center that is able to perform the step of the provision of an optimal medical facility and the step of the provision of location information and, optionally, the step of the provision of a stroke classification and, optionally, the step of the provision of a patient drop notification, and, optionally, additionally includes at least one element of the group comprising:
      a second device operated by a stroke specialist that is able to perform the step of the provision of a stroke classification; and
      at least one third device operated by at least one medical facility that is able to perform the step of the provision of a current occupancy rate.
      16. The system as claimed in claim 15,
      wherein the at least one first device is a mobile terminal or an in-vehicle data processing system of an emergency service vehicle and, additionally or alternatively,
      wherein the second device or the at least one third device is a mobile terminal or a stationary data processing system and, additionally or alternatively,
      wherein the central device is a stationary data processing system.

Although the invention has been illustrated and described in detail by the preferred embodiments, the invention is not limited by the disclosed examples and other variations can be derived herefrom by the person skilled in the art without departing from the scope of protection of the invention.

Even if not explicitly stated, individual example embodiments, or individual sub-aspects or features of these example embodiments, can be combined with, or substituted for, one other, if this is practical and within the meaning of the invention, without departing from the present invention. Without being stated explicitly, advantages of the invention that are described with reference to one example embodiment also apply to other example embodiments, where transferable.

Of course, the embodiments of the method according to the invention and the imaging apparatus according to the invention described here should be understood as being example. Therefore, individual embodiments may be expanded by features of other embodiments. In particular, the sequence of the method steps of the method according to the invention should be understood as being example. The individual steps can also be performed in a different order or overlap partially or completely in terms of time.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for provision of a stroke classification for a potential stroke patient in a medical information system including at least one local device and a central device including a data link with the at least one local device, the local device being associated with a potential stroke patient and the method comprising:
receiving patient identification information from the at least one local device at the central device, the patient identification information uniquely identifying the potential stroke patient in the medical information system;
querying patient information data from a database by the central device based on the patient identification information;
requesting medical information about the potential stroke patient from the at least one local device by the central device, the medical information including video data of at least the face of the potential stroke patient;
receiving at the central device, the medical information from the at least one local device; and
provisioning the stroke classification for the potential stroke patient based on the medical information and the patient information data.

2. The method of claim 1, further comprising:
transmitting the stroke classification to the at least one local device.

3. The method of claim 1, further comprising:
identifying, by the central device, specific data missing in the patient information data;
wherein the medical information includes the missing specific data identified.

4. The method of claim 3, wherein
the identifying step includes determining a predictive confidence value for the stroke classification for the potential stroke patient; and
identifying performed based upon the predictive confidence value.

5. The method of claim 1, further comprising:
identifying, by the central device, a medical facility for the potential stroke patient based at least on the stroke classification; and
provisioning, to the at least one local device, location information for the medical facility identified.

6. The method of claim 1, wherein the medical information further includes at least a stroke score that is derived from a patient test and wherein the requesting of the medical information additionally includes:
initiating recording of the video data of at least the face of the potential stroke patient; and
initiating performance of the patient test with the potential stroke patient and deriving of the stroke score.

7. The method of claim 1, wherein the provisioning of the stroke classification includes:
forwarding the medical information to a trained machine learning algorithm; and
autonomously predicting the stroke classification from the forwarded medical information by the trained machine learning algorithm, wherein the trained machine learning algorithm has been trained to predict stroke classifications from medical training information.

8. The method of claim 1, wherein the provisioning of the stroke classification encompasses:
transmitting the medical information to a stroke specialist for preparation of a provisional diagnosis as to whether the potential stroke patient has a stroke; and
deriving the stroke classification from a provisional diagnosis prepared based upon the medical information transmitted.

9. The method of claim 8,
wherein in the transmitting of the provided medical information, the medical information provided by the at least one local device is transmitted from the at least one local device to a second device, the second device being associated with the stroke patient, and
wherein in the deriving of the stroke classification, the stroke classification is derived by the second device.

10. The method of claim 9, wherein the requesting of the medical information additionally includes:
establishing a bidirectional communication link between the at least one local device and the second device, wherein the video data recorded of at least the face of the potential stroke patient is transmitted as an instantaneous transmission from the first device to the second device via the bidirectional communication link.

11. The method of claim 1, further comprising:
provisioning, in each case of patient information, data for multiple comparison patients, each comparison patient of the multiple comparison patients, being associated with a known stroke classification; and
identifying one or more reference patients from the multiple comparison patients based on similarity indicators, a similarity indicator, of the similarity indicators, is respectively based on a similarity between the patient information data for the potential stroke patient and the patient information data for a respective one comparison patient of the multiple comparison patients; and
wherein the stroke classification in the provisioning of the stroke classification is additionally provided based upon the known stroke classifications of the one or more reference patients.

12. A medical information system for provision of a stroke classification for a potential stroke patient, comprising:
a central device, configured to perform at least
receiving patient identification information from the at least one local device, the patient identification information uniquely identifying the potential stroke patient in the medical information system;
querying patient information data from a database based on the patient identification information;
requesting medical information about the potential stroke patient from the at least one local device, the medical information including video data of at least the face of the potential stroke patient;
receiving the medical information from the at least one local device; and
provisioning the stroke classification for the potential stroke patient based on the medical information and the patient information data.

13. The system as claimed in claim 12, further comprising:
the at least one local device, wherein the at least one local device is a mobile terminal or in-vehicle data processing system of an emergency service vehicle.

14. A non-transitory computer program product storing a program, directly loadable into a memory of a programmable processor of a central device, having program resources, to execute the method of claim 1 when the program is executed.

15. A non-transitory computer-readable storage medium storing readable and executable program sections to execute the method of claim 1 when the program sections are executed by a processing unit.

16. The method of claim 2, further comprising:
identifying, by the central device, specific data missing in the patient information data;
wherein the medical information includes the missing specific data identified.

17. The method of claim 16, wherein
the identifying step includes determining a predictive confidence value for the stroke classification for the potential stroke patient; and
identifying performed based upon the predictive confidence value.

18. A computer-readable storage medium storing readable and executable program sections to execute the method of claim 2 when the program sections are executed by a processing unit.

19. The method of claim 2, further comprising:
identifying, by the central device, a medical facility for the potential stroke patient based at least on the stroke classification; and
provisioning, to the at least one local device, location information for the medical facility identified.

20. The method of claim 2, wherein the medical information further includes at least a stroke score that is derived from a patient test and wherein the requesting of the medical information additionally includes:
initiating recording of the video data of at least the face of the potential stroke patient; and
initiating performance of the patient test with the potential stroke patient and deriving of the stroke score.

21. The method of claim 2, wherein the provisioning of the stroke classification includes:
forwarding the medical information to a trained machine learning algorithm; and
autonomously predicting the stroke classification from the forwarded medical information by the trained machine learning algorithm, wherein the trained machine learning algorithm has been trained to predict stroke classifications from medical training information.

22. The method of claim 10, wherein the patient test is performed by the stroke specialist with the potential stroke patient via the bidirectional communication link.

23. The medical information system of claim 12, wherein the central device includes,
at least one processor, configured to perform at least the querying of the patient information data; the requesting of the medical information; and the provisioning of the stroke classification.

24. The medical information system of claim 23, wherein the central device further includes,
an interface configured to perform the receiving of the patient identification information and the receiving of the medical information, from the at least one local device.

25. The medical information system of claim 12, wherein the central device includes,
at least one processor, configured to perform at least the querying of the patient information data; the requesting of the medical information; and the provisioning of the stroke classification.
